(19) United States
(12) Patent Application Publication
(10) Pub. No.: US 2020/0315595 A1
(43) Pub. Date: Oct. 8, 2020

(54) Grave DNA Extractor Assembly

(71) Applicants: David Wise, Bedford, TX (US); Nicolas Wise, Georgetown, TX (US); Sam Maner, Rockwood, TN (US)

(72) Inventors: David Wise, Bedford, TX (US); Nicolas Wise, Georgetown, TX (US); Sam Maner, Rockwood, TN (US)

(21) Appl. No.: 16/374,115

(22) Filed: Apr. 3, 2019

(51) Int. Cl. A61B 10/02; B23B 51/02; B01L 3/00; G01N 21/01

(52) U.S. Cl. CPC A61B 10/02; B23B 51/02; B01L 2200/141; G01N 21/01; B01L 3/5082

(57) ABSTRACT

A grave DNA extractor assembly for extracting DNA without unearthing bodies includes a drill, a grabbing assembly, a viewer, a tunnel tube, and a resealer. The drill is configured to drill through earth and a coffin. The grabbing assembly comprises a handle having a control mechanism. An extension is coupled to the handle and the control mechanism extends through the extension. Each of a plurality of grabber attachments is selectively engageable with the extension. The control mechanism opens and alternatively closes each of the plurality of grabber attachments to grab a DNA sample from within the coffin. The viewer comprises a camera, a light coupled to the camera, and a data cable coupled to the camera. The resealer is configured to patch the coffin.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to DNA extractors and more particularly pertains to a new DNA extractor for extracting DNA without unearthing bodies.

### BRIEF SUMMARY OF THE INVENTION

An embodiment of the disclosure meets the needs presented above by generally comprising a drill, a grabbing assembly, a viewer, a tunnel tube, and a resealer. The drill comprises a shaft having a plurality of shaft sections. Each of the plurality of shaft sections is selectively engageable with one another. The shaft is configured to attach to a drill. Each of a plurality of bits is selectively engageable with the shaft. The plurality of bits is configured to drill through earth and a coffin. The grabbing assembly comprises a handle having a control mechanism. An extension is coupled to the handle and the control mechanism extends through the extension. Each of a plurality of grabber attachments is selectively engageable with the extension and is in operational communication with the control mechanism of the handle. The control mechanism opens and alternatively closes each of the plurality of grabber attachments. The grabbing assembly is configured to grab a DNA sample from within the coffin. The viewer comprises a camera, a light coupled to the camera, and a data cable coupled to the camera. The data cable is configured to connect to a monitor or a personal electronic device. The tunnel tube receives the drill. The tunnel tube simultaneously receives the grabbing assembly and the viewer. The resealer is configured to patch the coffin.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a side elevation view of a grave DNA extractor assembly according to an embodiment of the disclosure.

FIG. 2 is an isometric view of an embodiment of the disclosure.

FIG. 3 is a detail view of an embodiment of the disclosure.

FIG. 4 is a detail view of an embodiment of the disclosure.

FIG. 5 is a detail view of an embodiment of the disclosure.

FIG. 6 is a detail view of an embodiment of the disclosure.

FIG. 7 is a detail view of an embodiment of the disclosure.

FIG. 8 is a detail view of an embodiment of the disclosure.

FIG. 9 is an in-use view of an embodiment of the disclosure.

FIG. 10 is an in-use view of an embodiment of the disclosure.

FIG. 11 is an in-use view of an embodiment of the disclosure.

FIG. 12 is an in-use view of an embodiment of the disclosure.

### DETAILED DESCRIPTION OF THE INVENTION

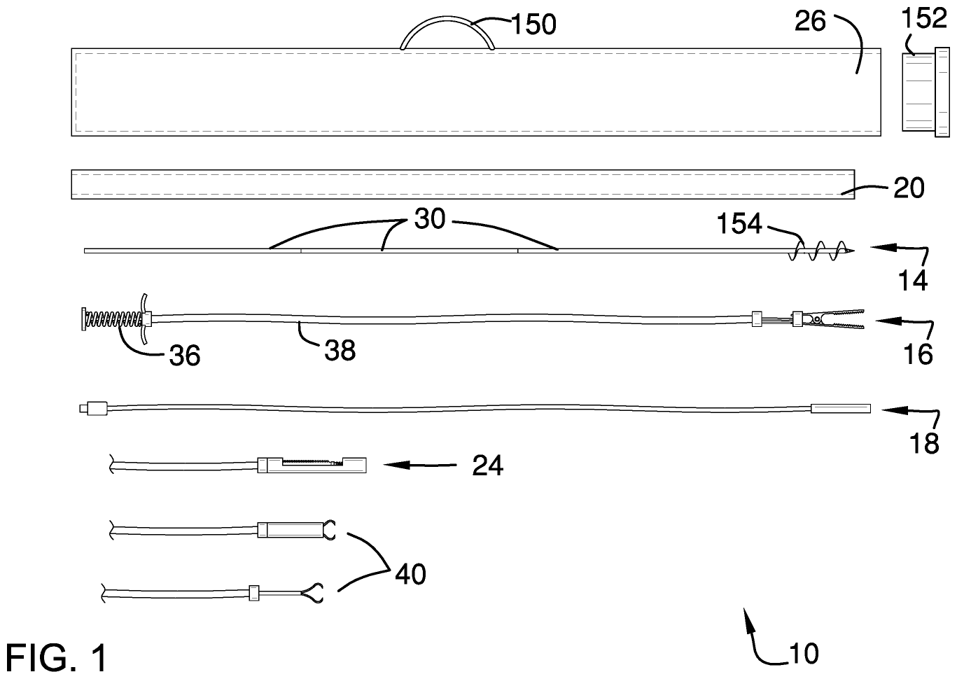

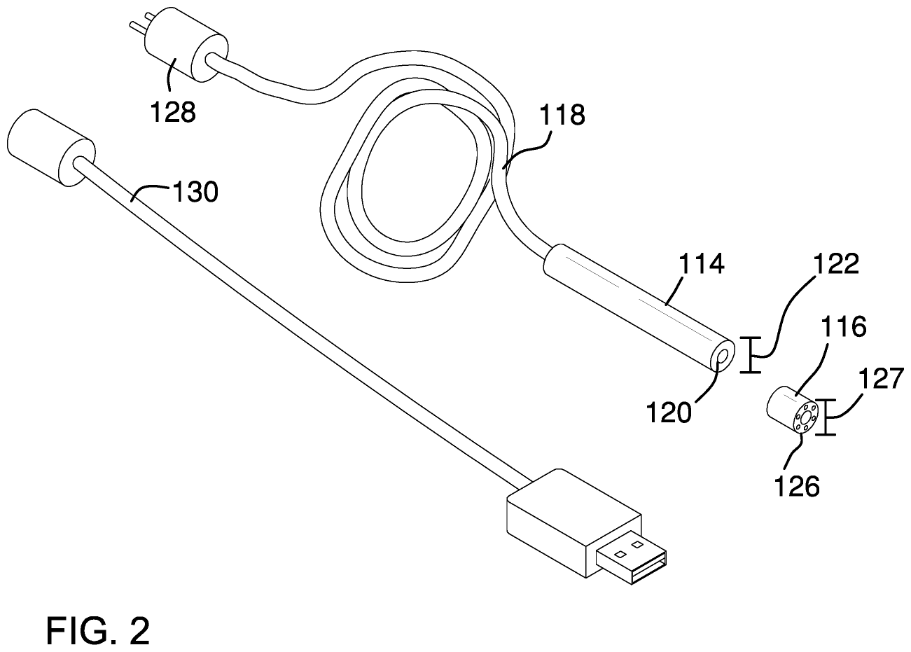

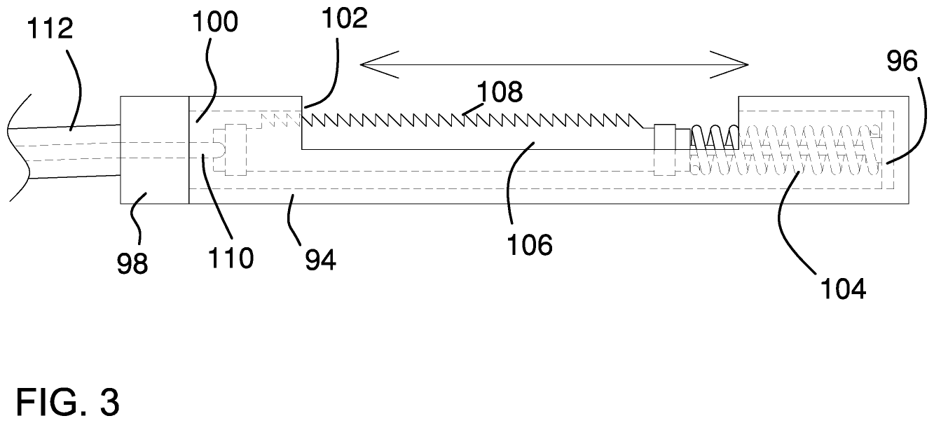

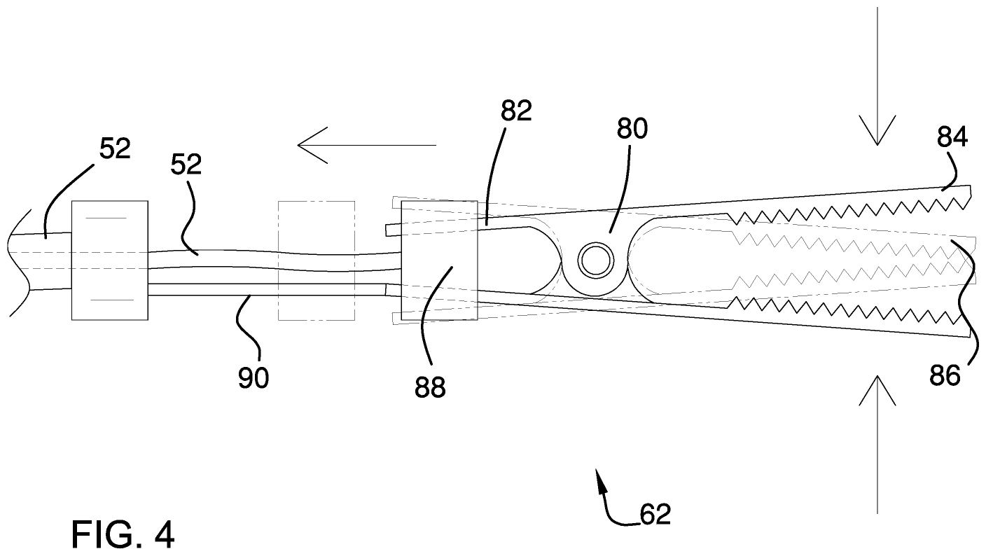

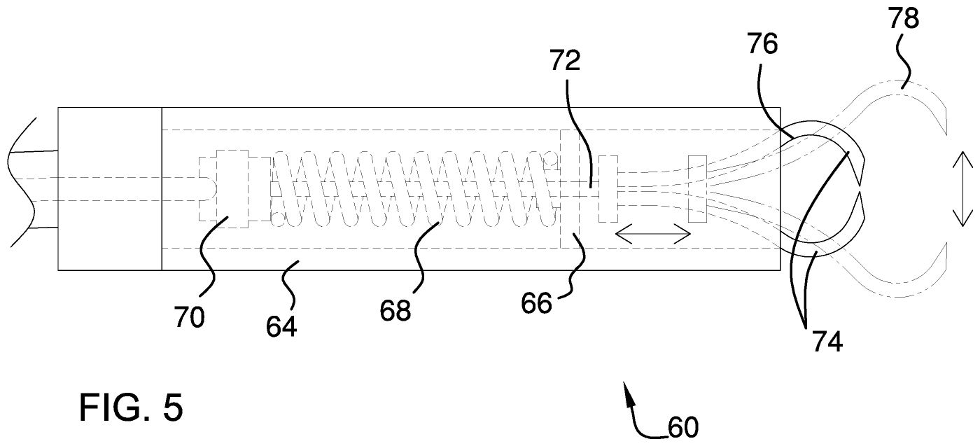

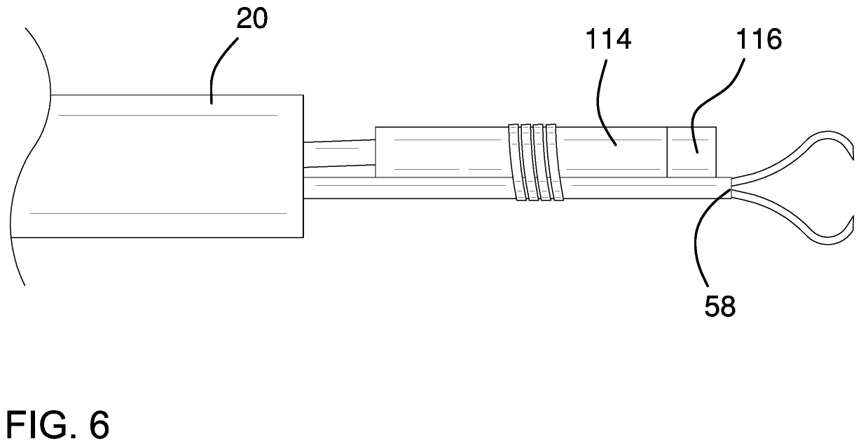

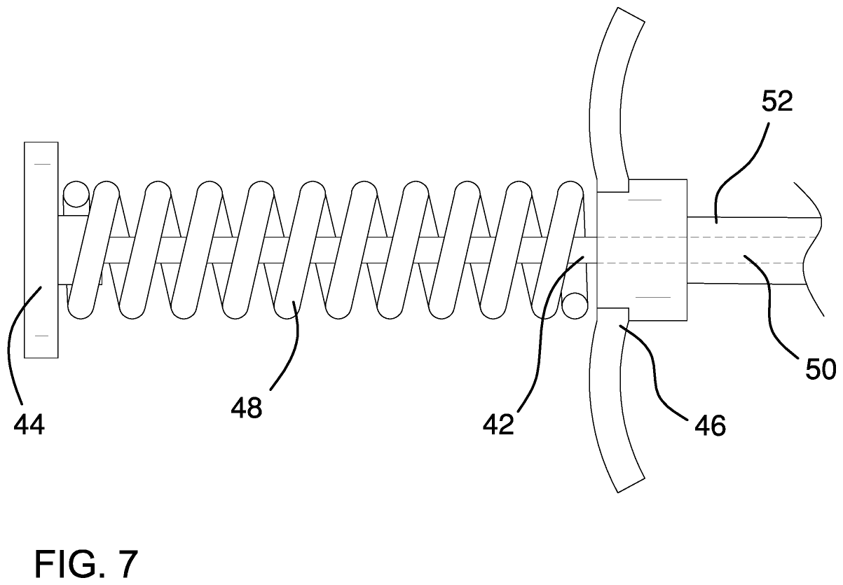

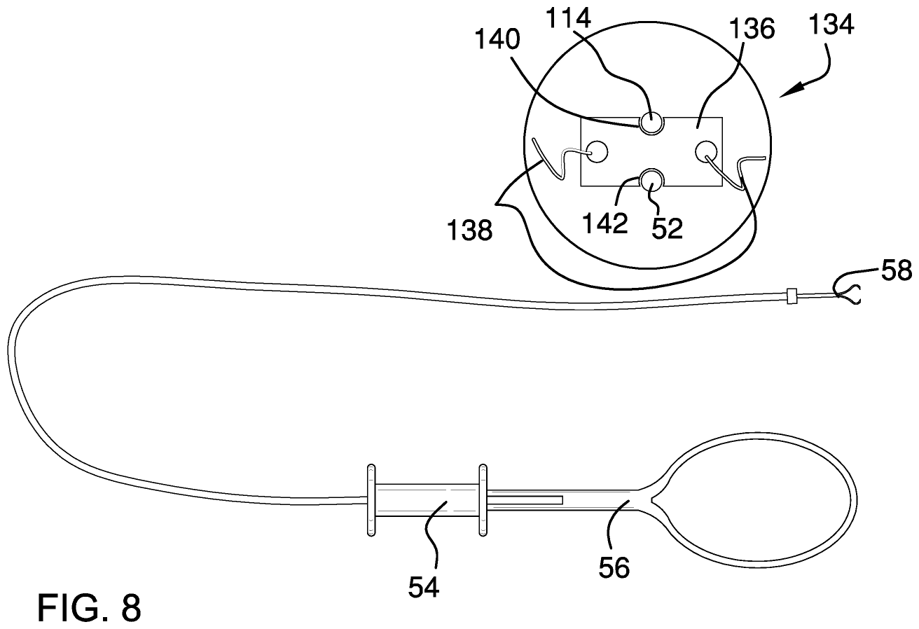

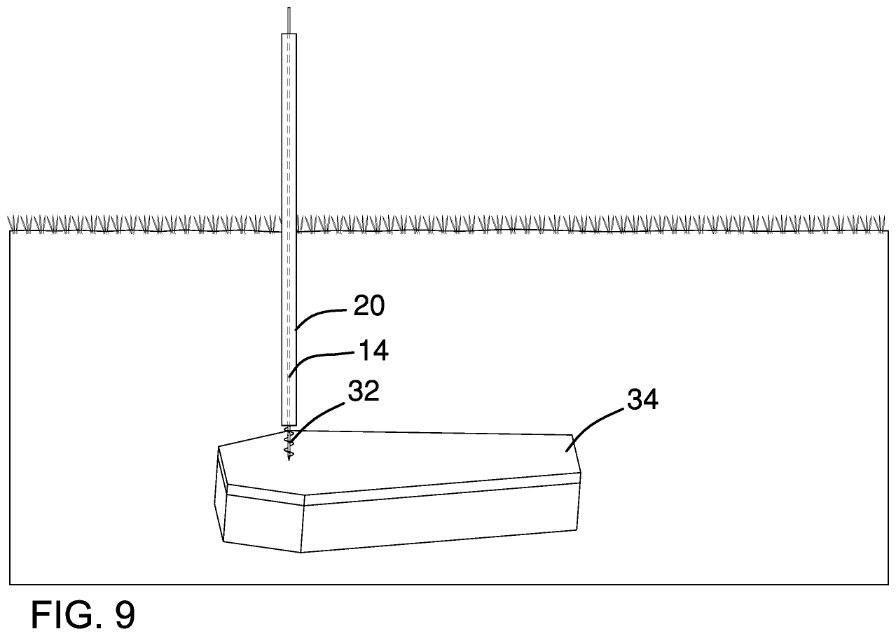

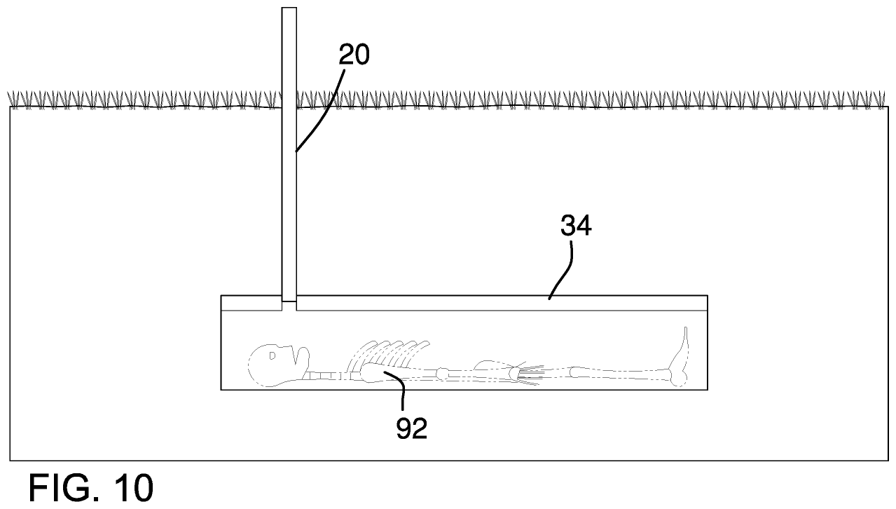

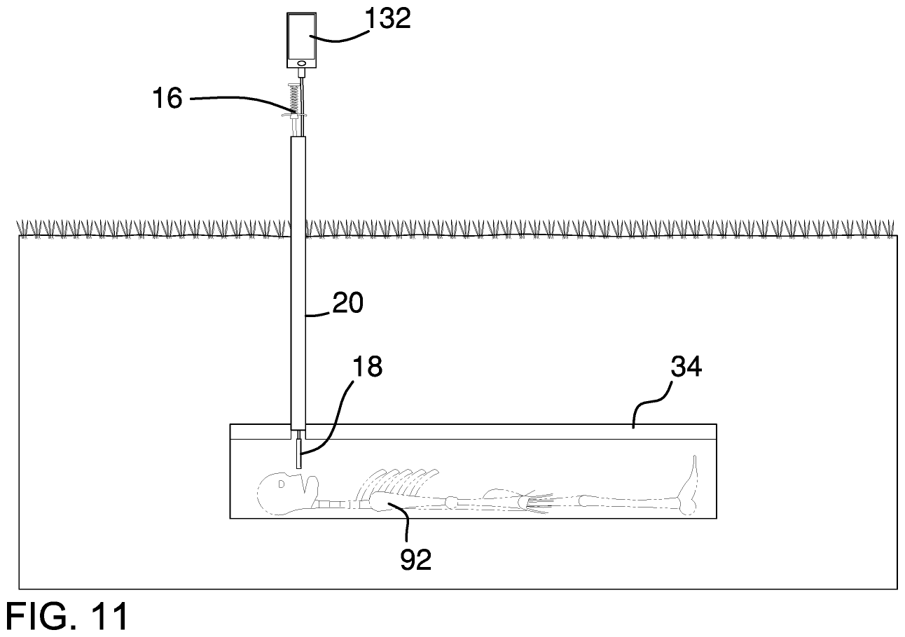

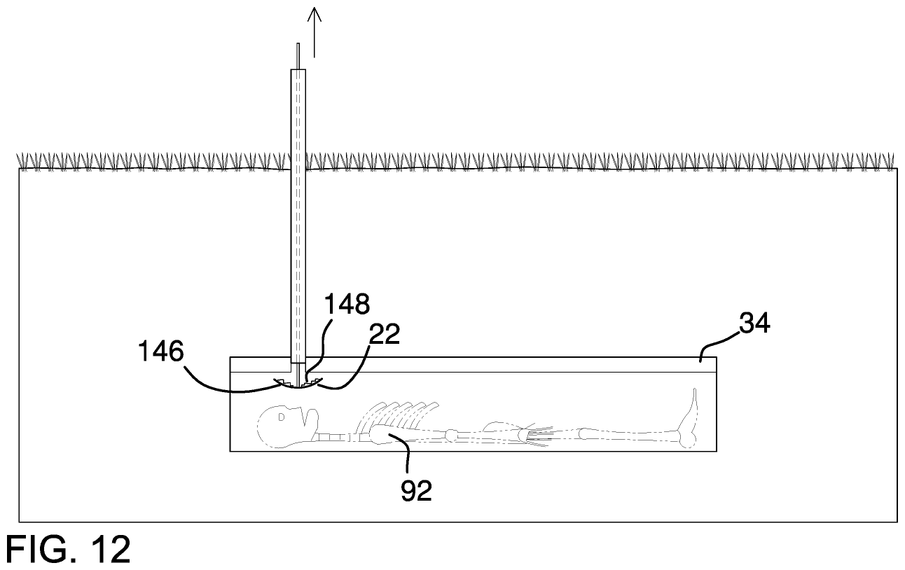

With reference now to the drawings, and in particular to FIGS. 1 through 12 thereof, a new DNA extractor embodying the principles and concepts of an embodiment of the disclosure and generally designated by the reference numeral 10 will be described.

As best illustrated in FIGS. 1 through 12, the grave DNA extractor assembly 10 generally comprises a drill 14, a grabbing assembly 16, a viewer 18, a tunnel tube 20, and a resealer 22. The grave DNA extractor assembly 10 may also comprise a saw 24 and a storage tube 26. The drill 14 comprises a shaft 28 having a plurality of shaft sections 30. Each of the plurality of shaft sections 30 is selectively engageable with one another. The shaft 28 is configured to attach to a drill. Each of a plurality of bits 32 is selectively engageable with the shaft 28. The plurality of bits 32 is configured to drill through earth and a coffin 34.

The grabbing assembly 16 comprises a handle 36, an extension 38, and a plurality of grabber attachments 40. The handle 36 may have a control mechanism 42, an end cap 44, a finger grip 46, and a handle spring 48. The control mechanism 42 may be a flex cable 50 coupled to the end cap 44. The handle spring 48 is coupled between the end cap 44 and the finger grip 46. The extension 38 may be a flex cable outer sheath 52 coupled to the finger grip 46. The flex cable 50 extends through the flex cable outer sheath 52. Each of the plurality of grabber attachments 40 is selectively engageable with the extension 38 and is in operational communication with the control mechanism 42 of the handle. The control mechanism 42 opens and alternatively closes each of the plurality of grabber attachments 40 by compressing the end cap 44 towards the finger grip 46. The handle 36 may alternatively comprise a grip section 54 with the controller being a pull loop 56 extending through the grip section 54 to engage the flex cable 50. The plurality of grabber attachments 40 comprises a standard grab claw 58, a spring-loaded grab claw 60, and an alligator clip claw 62. The spring-loaded grab claw 58 comprises a tubular housing 64. The tubular housing 64 is selectively engageable with the extension 38 of the grabbing assembly. A stop plate 66 is coupled within the tubular housing 64 and a compression spring 68 is coupled to the stop plate 66. The compression spring 68 has an attachment end 70 that is selectively engageable with the control mechanism 42 of the grabbing assembly. A claw rod 72 is coupled to the attachment end 70 and coaxially extends through the compression spring 68 and the stop plate 66. A pair of claws 74 is coupled to the claw rod 72. The pair of claws 74 has a retracted position 76 and an alternate extended position 78. The pair of claws 74 is forced shut by the tubular housing 64 in the retracted position, the pair of claws 74 is open in the alternate extended position 78. The compression spring 68 automatically returns the pair of claws 74 from the alternate extended position 78 to the retracted position 76. The alligator clip claw 62 comprises an alligator clip 80 having a pinch end 82, an open position 84, and an alternate closed position 86. A collar 88 is selectively engageable with the control mechanism 42 of the grabbing assembly and is selectively engageable with the pinch end 82 of the alligator clip to maintain the open position 84. The control mechanism 42 is used to remove the collar 88 from the alligator clip 80 to move to the alternate closed position 86. A clip holder 90 is coupled to the alligator clip 80. The clip holder 90 is selectively engageable with the extension 38 of the grabbing assembly. The grabbing assembly 16 is configured to grab a DNA sample from a corpse 92 within the coffin 34.

The saw 24 comprises a saw housing 94 having a distal end 96, a proximal end 98, an inside 100, and a channel 102 extending through to the inside 100. A return spring 104 is coupled within the inside 100. A saw blade 106 is coupled to the return spring 104. The saw blade 106 has a toothed side 108 that aligns with the channel 102 of the saw housing. A push rod 110 is coupled to the saw blade 106 and is slidably coupled through the proximal end 98 of the saw housing. A rod sheath 112 is coupled to the saw housing 94. The rod sheath 112 is coaxially disposed around the push rod 110 and is coupled to the proximal end 98 of the saw housing. The push rod 110 is used to move the saw blade 106 to compress the return spring 104, then the return spring 104 moves the saw blade 106 back when the pressure is released from the push rod 110 thus causing the saw blade 106 to reciprocate and cut.

The viewer 18 comprises a camera 114, a light 116, and a data cable 118. The camera 114 has a lens 120 and may be cylindrical with a first diameter 122. The light 116 is coupled to the camera 114 and may be cylindrical with a second diameter 124 substantially conforming to the first diameter 122. The light 116 has a through hole 126 to allow for the lens 120 to see. The data cable 118 is coupled to the camera 114 and may have a plug 128 that selectively engages an adaptor 130. The adaptor 130 is configured to connect to a monitor or a personal electronic device 132 and may be a USB adaptor. There may be a claw camera clip 134 to join the viewer 18 and the grabbing assembly 116. The claw camera clip 134 has a clip body 136 and a pair of guide wires 138. The clip body 136 has a camera slot 140 and a cable slot 142 extending through the clip body 136. The camera slot 140 selectively receives the camera 114 and the cable slot 142 selectively receives the flex cable outer sheath 52. The clip body 136 is coupled to the pair of guide wires 138.

The tunnel tube 20 receives the drill 14 and simultaneously receives the grabbing assembly 16 and the viewer 18. The resealer 22 may comprise a screen 146 having a resin side 148. The resin side 148 selectively engages with the shaft 28. The storage tube 26 has a carry handle 150 and an end cap 152. The end cap 152 seals and alternatively unseals the storage tube 26. The storage tube 26 receives the drill 14, the grabbing assembly 16, the viewer 18, the tunnel tube 20, the saw 24, and the resealer 22.

In use, the drill 14 is used with an auger drill bit 154 to dig through the earth until reaching the coffin 34 and then remove the drill. The tunnel tube 20 is then placed in the hole and the drill 14 is placed through the tunnel tube with a different drill bit of the plurality of bits 32 to drill through the coffin 34. The drill 14 is then removed and the viewer 18 and the grabbing assembly 16 are simultaneously placed through the tunnel tube 20 to extract the DNA sample. The viewer 18 and the grabbing assembly 16 are removed and the resealer 22 is then placed in the tunnel tube 20. The shaft 28 is used to push the screen 146 through the tunnel tube 20 and into the coffin 34. The shaft 28 is then lifted back out and the resin side 148 seals the coffin 34.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A grave DNA extractor assembly, the assembly comprising:
a drill, the drill comprising:
a shaft, the shaft having a plurality of shaft sections, each of the plurality of shaft sections being selectively engageable with one another, the shaft being configured to attach to a drill; and
a plurality of bits, each of the plurality of bits being selectively engageable with the shaft, the plurality of bits being configured to drill through earth and a coffin;
a grabbing assembly, the grabbing assembly comprising:
a handle, the handle having a control mechanism;
an extension coupled to the handle, the control mechanism extending through the extension; and
a plurality of grabber attachments, each of the plurality of grabber attachments being selectively engageable with the extension and being in operational communication with the control mechanism of the handle, the control mechanism opening and alternatively closing each of the plurality of grabber attachments;
wherein the grabbing assembly is configured to grab a DNA sample from within the coffin;
a viewer, the viewer comprising:
a camera;
a light coupled to the camera; and
a data cable coupled to the camera, the data cable being configured to connect to a monitor or a personal electronic device;
a tunnel tube, the tunnel tube receiving the drill, the tunnel tube simultaneously receiving the grabbing assembly and the viewer; and
a resealer, the resealer being configured to patch the coffin.

2. The grave DNA extractor assembly of claim 1 further comprising a saw, the saw comprising:
a saw housing, the saw housing having a distal end, a proximal end, an inside, and a channel extending through to the inside;
a return spring coupled to the saw housing, the return housing being coupled within the inside;
a saw blade being coupled to the return spring, the saw blade having a toothed side aligning with the channel of the saw housing;
a push rod coupled to the saw blade, the pushrod being slidably coupled through the proximal end of the saw housing;
a rod sheath coupled to the saw housing, the rod sheath being coaxially disposed around the push rod and coupled to the proximal end of the saw housing;
wherein the push rod is used to move the saw blade to compress the return spring, the return spring moving the saw blade back when the pressure is released from the push rod, the saw blade thus reciprocating.

3. The grave DNA extractor assembly of claim 1 further comprising the control mechanism of the handle of the grabbing assembly being a flex cable, the extension being a flex cable outer sheath, the flex cable extending through the flex cable outer sheath to be in operational communication with the plurality of grabber attachments, the handle having an end cap coupled to the flex cable, a finger grip coupled to the flex cable outer sheath, and a handle spring coupled between the end cap and the finger grip, the plurality of grabber attachments being controlled by compressing the end cap towards the finger grip.

4. The grave DNA extractor assembly of claim 1 further comprising the plurality of grabber attachments comprising a standard grab claw, a spring-loaded grab claw, and an alligator clip claw.

5. The grave DNA extractor assembly of claim 4 further comprising the spring-loaded grab claw comprising:
a tubular housing, the tubular housing being selectively engageable with the extension of the grabbing assembly;
a stop plate coupled within the tubular housing;
a compression spring coupled to the stop plate, the compression spring having an attachment end, the attachment end being selectively engageable with the control mechanism of the grabbing assembly;
a claw rod coupled to the attachment end, the claw rod coaxially extending through the compression spring and the stop plate; and
a pair of claws coupled to the claw rod, the pair of claws having a retracted position and an alternate extended position, the pair of claws being forced shut by the tubular housing in the retracted position, the pair of claws being open in the alternate extended position, the compression spring automatically returning the pair of claws from the alternate extended position to the retracted position.

6. The grave DNA extractor assembly of claim 4 further comprising the alligator clip claw comprising:
an alligator clip, the alligator clip having a pinch end, an open position, and an alternate closed position;
a collar, the collar being selectively engageable with the control mechanism of the grabbing assembly, the collar being selectively engageable with the pinch end of the alligator clip to maintain the open position, the control mechanism being used to remove the collar from the alligator clip to move to the alternate closed position; and
a clip holder, the clip holder being coupled to the alligator clip, the clip holder being selectively engageable with the extension of the grabbing assembly.

7. The grave DNA extractor assembly of claim 1 further comprising the camera being cylindrical and having a first diameter, the light being cylindrical and having a second diameter substantially conforming to the first diameter, the light having a through hole to allow for a lens of the camera to see.

8. The grave DNA extractor assembly of claim 7 further comprising the data cable of the camera having a plug, the plug selectively engaging an adaptor, the adaptor being configured to connect to the monitor or the personal electronic device.

9. The grave DNA extractor assembly of claim 3 further comprising a claw camera clip, the claw camera clip having a clip body, and a pair of guide wires, the clip body having a camera slot and a cable slot extending through the clip body, the camera slot selectively receiving the camera, the cable slot selectively receiving the flex cable outer sheath, the clip body being coupled to a pair of guide wires.

10. The grave DNA extractor assembly of claim 1 further comprising the resealer comprising a screen, the screen having a resin side, the resin side selectively engaging with the shaft, the shaft being used to push the screen through the tunnel tube and into the coffin, the shaft then being lifted back out and the resin side being configured to seal the coffin.

11. The grave DNA extractor assembly of claim 1 further comprising the handle having a grip section, the controller being a pull loop extending through the grip section.

12. The grave DNA extractor assembly of claim 2 further comprising a storage tube, the storage tube having a carry handle and an end cap, the end cap sealing and alternatively unsealing the storage tube, the storage tube receiving the drill, the grabbing assembly, the viewer, the tunnel tube, the saw, and the resealer.

13. A grave DNA extractor assembly, the assembly comprising:
a drill, the drill comprising:
a shaft, the shaft having a plurality of shaft sections, each of the plurality of shaft sections being selectively engageable with one another, the shaft being configured to attach to a drill; and
a plurality of bits, each of the plurality of bits being selectively engageable with the shaft, the plurality of bits being configured to drill through earth and a coffin;
a grabbing assembly, the grabbing assembly comprising:
a handle, the handle having a control mechanism, an end cap, a finger grip, and a handle spring, the control mechanism being a flex cable, the end cap being coupled to the flex cable, the handle spring being coupled between the end cap and the finger grip;
an extension coupled to the handle, the extension being a flex cable outer sheath coupled to the finger grip, the flex cable extending through the flex cable outer sheath; and
a plurality of grabber attachments, each of the plurality of grabber attachments being selectively engageable with the extension and being in operational communication with the control mechanism of the handle, the control mechanism opening and alternatively closing each of the plurality of grabber attachments by compressing the end cap towards the finger grip, the plurality of grabber attachments comprising:
a standard grab claw;
a spring-loaded grab claw, the spring-loaded grab claw comprising:
a tubular housing, the tubular housing being selectively engageable with the extension of the grabbing assembly;
a stop plate coupled within the tubular housing;
a compression spring coupled to the stop plate, the compression spring having an attachment end, the attachment end being selectively engageable with the control mechanism of the grabbing assembly;
a claw rod coupled to the attachment end, the claw rod coaxially extending through the compression spring and the stop plate; and
a pair of claws coupled to the claw rod, the pair of claws having a retracted position and an alternate extended position, the pair of claws being forced shut by the tubular housing in the retracted position, the pair of claws being open in the alternate extended position, the compression spring automatically returning the pair of claws from the alternate extended position to the retracted position; and
an alligator clip claw, the alligator clip claw comprising:
an alligator clip, the alligator clip having a pinch end, an open position, and an alternate closed position;
a collar, the collar being selectively engageable with the control mechanism of the grabbing assembly, the collar being selectively engageable with the pinch end of the alligator clip to maintain the open position, the control mechanism being used to remove the collar from the alligator clip to move to the alternate closed position; and
a clip holder, the clip holder being coupled to the alligator clip, the clip holder being selectively engageable with the extension of the grabbing assembly;
wherein the grabbing assembly is configured to grab a DNA sample from within the coffin;
a saw, the saw comprising:
a saw housing, the saw housing having a distal end, a proximal end, an inside, and a channel extending through to the inside;
a return spring coupled to the saw housing, the return housing being coupled within the inside;
a saw blade being coupled to the return spring, the saw blade having a toothed side aligning with the channel of the saw housing;
a push rod coupled to the saw blade, the pushrod being slidably coupled through the proximal end of the saw housing;
a rod sheath coupled to the saw housing, the rod sheath being coaxially disposed around the push rod and coupled to the proximal end of the saw housing;
wherein the push rod is used to move the saw blade to compress the return spring, the return spring moving the saw blade back when the pressure is released from the push rod, the saw blade thus reciprocating;
a viewer, the viewer comprising:
a camera, the camera having a lens, the camera being cylindrical and having a first diameter;
a light coupled to the camera, the light being cylindrical and having a second diameter substantially conforming to the first diameter, the light having a through hole to allow for the lens to see; and
a data cable coupled to the camera, the data cable having a plug, the plug selectively engaging an adaptor, the adaptor being configured to connect to a monitor or a personal electronic device;
a claw camera clip, the claw camera clip having a clip body, and a pair of guide wires, the clip body having a camera slot and a cable slot extending through the clip body, the camera slot selectively receiving the camera, the cable slot selectively receiving the flex cable outer sheath, the clip body being coupled to a pair of guide wires;
a tunnel tube, the tunnel tube receiving the drill, the tunnel tube simultaneously receiving the grabbing assembly and the viewer;
a resealer, the resealer comprising a screen, the screen having a resin side, the resin side selectively engaging with the shaft, the shaft being used to push the screen through the tunnel tube and into the coffin, the shaft then being lifted back out and the resin side being configured to seal the coffin; and
a storage tube, the storage tube having a carry handle and an end cap, the end cap sealing and alternatively unsealing the storage tube, the storage tube receiving the drill, the grabbing assembly, the viewer, the tunnel tube, the saw, and the resealer.
